(19) United States
(12) Patent Application Publication
(10) Pub. No.: US 2005/0208145 A1
(43) Pub. Date: Sep. 22, 2005

(54) Grain fiber compositions and methods of use

(76) Inventors: Thava Vasanthan, Edmonton (CA); Feral Temelli, Edmonton (CA)

Correspondence Address: SQUIRE, SANDERS & DEMPSEY L.L.P., 1 MARITIME PLAZA, SUITE 300, SAN FRANCISCO, CA, 94111, US

(21) Appl. No.: 10/804,058

(22) Filed: Mar. 19, 2004

(51) Int. Cl. A61K 9/16; A61K 9/50

(52) U.S. Cl. 424/490; 536/123.12

(57) ABSTRACT

The present invention relates to beta-glucan compositions having a honeycomb structure and the uses of the compositions as encapsulating agents.

This application claims the benefit of priority from U.S. patent application Ser. No. 10/397,215 filed Mar. 27, 2003.

### FIELD OF THE INVENTION

The present invention relates to beta-glucan compositions having a honeycomb structure and the uses of the compositions as micro-encapsulating agents.

### BACKGROUND OF THE INVENTION

Encapsulation is a process where one material, an encapsulant, is entrapped within a coating material to form a capsule. The resulting capsule can be used in numerous applications including, for example, to protect the encapsulant from a specific environment until a desired time, location or condition is reached upon which the structure of the coating material disintegrates and the encapsulant is released. Encapsulation technology is used in various industries including the drug, food ingredients and cosmetics industries, usually to ensure that an encapsulated drug or composition is protected until a desired location, time or condition for the release of the encapsulant is reached.

Encapsulating media for the delivery of drugs to the digestive tract are well known. Generally, the properties of the coating or wall protect the encapsulant from physical loss, degradation of the encapsulant by oxidation or otherwise provides protection from a surrounding environment. As such, the properties of the coating or wall in relation to the environment to which the coating may be exposed are determinative of the manner in which the encapsulant is released. Accordingly, for particular applications it is desirable that the specific properties of the coating that enable its disintegration be adapted for the specific time, location or condition goals of encapsulant release.

Capsules are generally classified according to their particle size such as macro (having a particle size diameter >5000 microns), micro (having a particle size diameter 0.2-5000 microns) and nano (having a particle size diameter <0.2 microns). Different capsule sizes have found various applications. For example, micro encapsulation has found a variety of food and non-food industrial applications. Two of the most commonly used micro encapsulation techniques are extrusion and spray drying as described in Dziezak, Food Technology 42:136, 1988.

In particular, modified starches and dextrins/cyclodextrins are commonly used as a wall material for encapsulation. Although, these wall materials protect compounds from degradation (i.e. oxidative, photo, etc), once ingested, they become quickly solubilized in intestinal fluid and digested by intestinal amylase enzymes. The thickness of the wall material can be designed to adjust the time of release to provide some flexibility in the controlled release of compounds in the human/animal intestinal tract.

Other materials, such as lipid-based wall materials (such as mono- and diglycerides) allow the release of encapsulant upon melting of the wall material at a certain temperature.

Isolated cell walls from barley and oat grain (whole or pearled), which are rich in beta glucans, have not been used as a wall material for microencapsulation. However, beta glucans have unique properties (as illustrated below) in comparison to starches, dextrins and cyclodextrins within the human intestinal tract such that they are an attractive coating material.

Some of the particular properties of beta-glucans include i) adjustable solubility (depending on the processing parameters used during its isolation from grains) at body temperature within the middle/distal regions of the human intestinal tract as compared to starch, ii) the absence of enzymes in the frontal region of the human intestinal tract that can digest beta-glucan, iii) the micro-flora at the distal region of the human intestinal tract can ferment and digest beta-glucan, iv) beta-glucan is a neutraceutical and v) beta-glucan is a very low caloric compound.

As a result, the use of beta-glucans for encapsulation is attractive where the absorption or release of particular compounds is desired in the lower intestinal tract. Thus, there has been a need for beta-glucans as encapsulating materials and, in particular, for encapsulating materials that include preserved cell wall structures of grain flours.

Furthermore, the isolation of intact cell walls from other cereals (eg. wheat, rye, rice, etc) and legumes (field pea, lentil, chick peas etc) are possible. These cell walls are primarily composed of water insoluble components (i.e. cellulose and hemi-cellulose) that vary in their solubility characteristics and thus will improve the flexibility in controlled release of compounds in certain applications.

A review of the prior art reveals that beta-glucans have not been previously utilized for coatings.

For example, U.S. Pat. No. 6,562,459 discloses a method to produce microspheres from water-insoluble cereal grain polysaccharides (starches). The polysaccharides are dissolved in a solvent and a precipitant is added to allow formation and collection of the microspheres. Suitable solvents include DMSO, formamide, acetamide, or aqueous solutions with high or low pH, and suitable precipitants include water, dichloromethane, and alcohol/water mixtures. The microspheres produced are of nm-100 um in diameter, with a spherical deviation of up to 25%. The microspheres can be used for various purposes, including vehicles for delivering active substances in pharmaceutical applications, as encapsulating materials, as vehicles for slow release of active substances, etc, and are stated to be biocompatible and biodegradable, being particularly advantageous for use in humans or animals. This method uses alpha-linked glucose polymers (starch, amylose and amylopectin, from plant sources, glycogen from animal sources and dextran from microbial sources) and a blend of linear and branched polymers.

Other references include U.S. Patent Application No. 2002/0164374, which discloses a biodegradable insoluble polymer which changes phase between 25 degrees Celsius and 37 degrees Celsius to become a liquid, the polymer for use as a drug delivery system; U.S. Pat. No. 6,569,463 and U.S. Pat. No. 6,248,363, which disclose an encapsulation coat and an active agent, held together by a solid carrier; U.S. Pat. No. 5,573,783, which discloses a formulation for delivery of a low solubility drug, in which a carrier is coated with nanoparticles of the drug; U.S. Pat. No. 5,534,270, which discloses a method of preparing sterilized nanoparticulate crystalline drug particles; U.S. Pat. No. 6,624,300 and U.S. Pat. No. 6,323,338, which disclose an aqueous method to concentrate beta-glucan in the form of a film; and, U.S. Pat. No. 6,500,463, which discloses an encapsulation system including a plasticizable matrix material mixed with a liquid plasticizer.

### SUMMARY OF THE INVENTION

In accordance with one embodiment of the invention, there is provided a beta-glucan concentrate having a honeycomb structure.

The invention also provides a method of preparing a beta-glucan (BG) concentrate product comprising the steps of: a) mixing a flour and an alcohol to form a flour/alcohol slurry; b) separating a fiber residue from the alcohol, wherein the fiber residue has a high BG content; and c) subjecting the fiber residue from step b) to at least one additional treatment step, the additional treatment step including mixing the fiber residue from step b) with an alcohol to form a fiber residue/alcohol slurry and subjecting the fiber residue/alcohol slurry to a sonication, protease or amylase treatment step or a combination of a sonication, protease or amylase treatment steps and thereafter separating a final concentrate from the fiber residue/alcohol slurry wherein the fiber concentrate has a honeycomb structure.

In a still further embodiment, the invention provides the use of a beta-glucan concentrate having a honeycomb structure as wall material for encapsulation.

In yet a still further embodiment, the invention provides a method of encapsulating a compound within a beta-glucan concentrate having a honeycomb structure comprising the steps of: mixing a beta-glucan concentrate having a honeycomb structure with an encapsulant under conditions to promote inclusion of the encapsulant within the honeycomb structure; and washing the resulting capsules.

In further embodiments of the preceding method, the honeycomb structure may be partially solubilized during mixing. Furthermore, the mixing step may be an extrusion process and the honeycomb structure may be subjected to a pre-mixing step wherein the beta-glucan concentrate is moistened with water (preferably 15-40% (w/w)). The resulting capsules may also be sealed to form a sealed honeycomb structure.

In a further embodiment, the invention provides a method of encapsulating a compound within a beta-glucan concentrate having a honeycomb structure comprising the steps of: mixing a beta-glucan concentrate having a honeycomb structure with an encapsulant under conditions to promote partial solubilization of the beta-glucan concentrate; and, adjusting the conditions of mixing to precipitate beta-glucan concentrate.

### BRIEF DESCRIPTION OF THE DRAWINGS

The invention is described with reference to the following drawings wherein:

FIG. 1A is a scanning electron micrograph of barley flour;

FIG. 1B is a scanning electron micrograph of lab-processed beta-glucan concentrate obtained from barley flour showing a honeycomb structure in accordance with the invention;

FIG. 2A is a scanning electron micrograph of oat flour;

FIG. 2B is a scanning electron micrograph of lab-processed beta-glucan concentrate obtained from oat flour showing a honeycomb structure in accordance with the invention;

FIG. 3 are two scanning electron micrographs of pilot plant processed beta-glucan concentrate obtained from barley flour showing a honeycomb structure in accordance with the invention;

FIG. 4 are two scanning electron micrographs of pilot plant processed beta-glucan concentrate obtained from oat flour showing a honeycomb structure in accordance with the invention; and,

FIG. 5 is a flow chart a methodology of encapsulation in accordance with the invention.

### DETAILED DESCRIPTION OF THE INVENTION

With reference to the Figures, unique structures of fiber concentrates enriched in beta-glucan and processes for encapsulation using the unique beta-glucan structures are described.

As described in Applicant's co-pending patent application, U.S. patent application Ser. No. 10/397,215 (incorporated herein by reference), methods of preparing high viscosity beta-glucan products and high viscosity fiber concentrates are described. In particular, Applicant's co-pending application describes a method of preparing a beta-glucan (BG) concentrate product comprising the steps of:
a) mixing a flour and an alcohol to form a flour/alcohol slurry;
b) separating a fiber residue from the alcohol, wherein the fiber residue has a high BG content; and,
c) subjecting the fiber residue from step b) to at least one additional treatment step, the additional treatment step including mixing the fiber residue from step b) with an alcohol to form a fiber residue/alcohol slurry and subjecting the fiber residue/alcohol slurry to a sonication, protease or amylase treatment step or a combination of a sonication, protease or amylase treatment step and thereafter separating a final fiber concentrate from the fiber residue/alcohol slurry.

The high viscosity fiber concentrates prepared by utilizing the Applicant's methodology have been further characterized through examination of the structures of the product utilizing scanning electron microscopy (SEM).

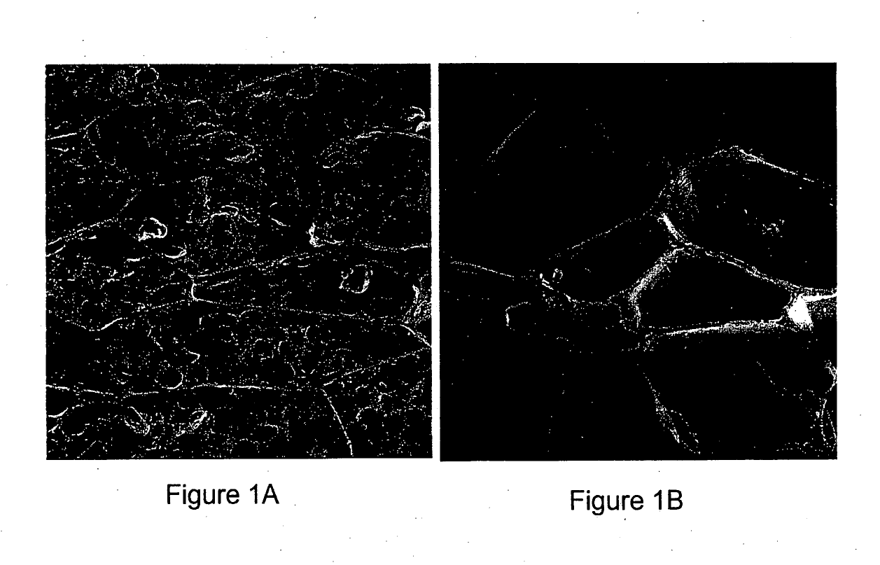

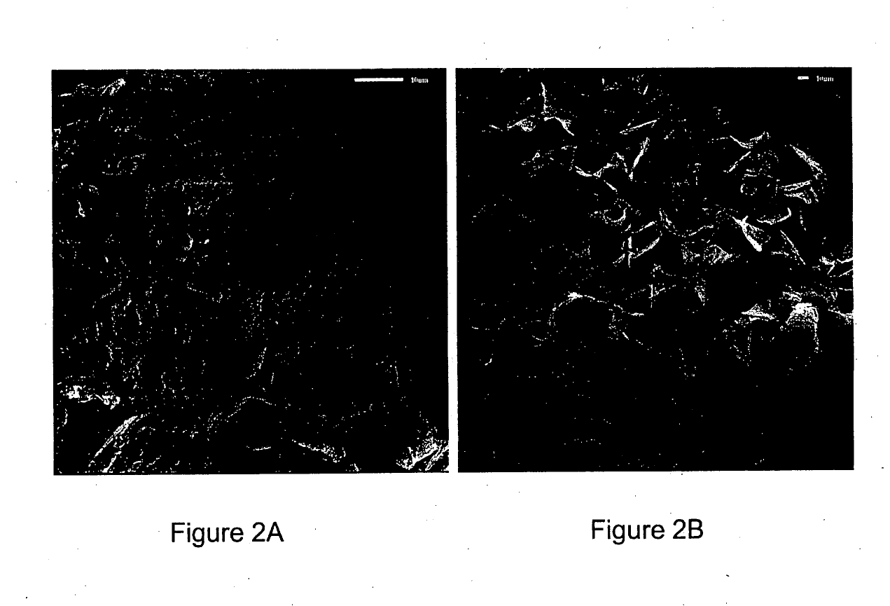

With reference to FIGS. 1A, 1B, 2A, 2B, 3 and 4, a comparison of the structures of untreated barley and oat flours (FIGS. 1A and 2A, respectively) and the structures of the beta-glucan concentrates prepared from each pearled grain flour (FIGS. 1B, 2B and 3 and 4, respectively) in accordance with Applicant's methodology are shown for both lab and pilot plant based processes.

As shown in FIGS. 1A and 1B for barley, the untreated barley flour of FIG. 1A shows the intact flour particles which include the cell wall structure as well as the cell contents. The intact cell wall structure is comprised mainly of beta-glucan, and some hemi-cellulose and cellulose and the interior of the cells contains predominantly starch granules embedded in a protein matrix. Following treatment in accordance with the above methodology, FIG. 1B shows essentially an intact cell wall structure of the flour in which the starch and protein components within the cells have been reduced (i.e. the cell contents have been removed) resulting in a cell wall/fiber concentrate that is enriched in beta glucan having a honeycomb structure. Similar results are shown in FIGS. 2A and 2B for oat flour and oat beta glucan concentrate.

As is evident from the electron micrographs, the treated flours produce a honeycomb structure having significant void space within an essentially intact cell wall structure. Accordingly, a honeycomb structure is herein defined as a concentrate of beta glucan which comprises an essentially intact cell wall structure or matrix of the starting flour and that has significant void spaces within the cell wall structure or matrix.

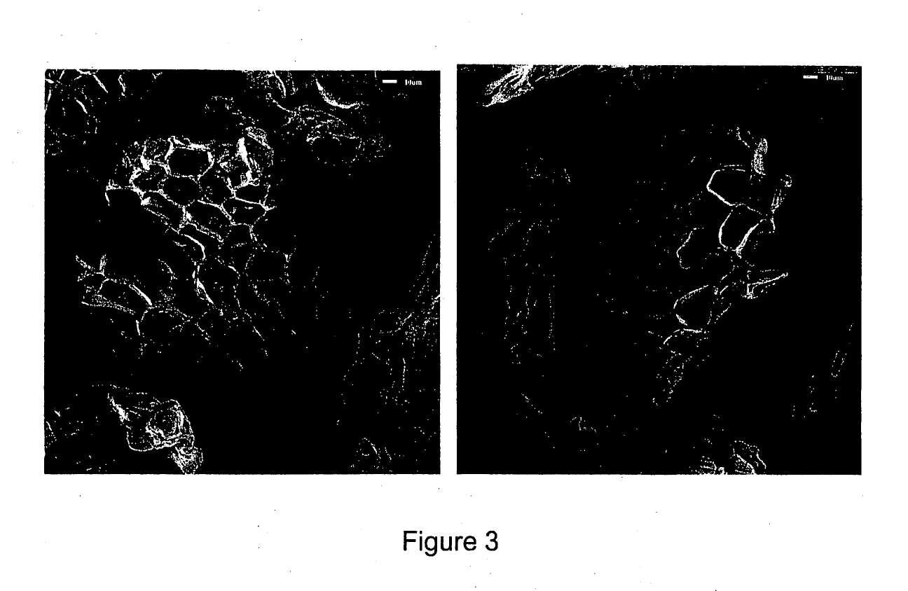

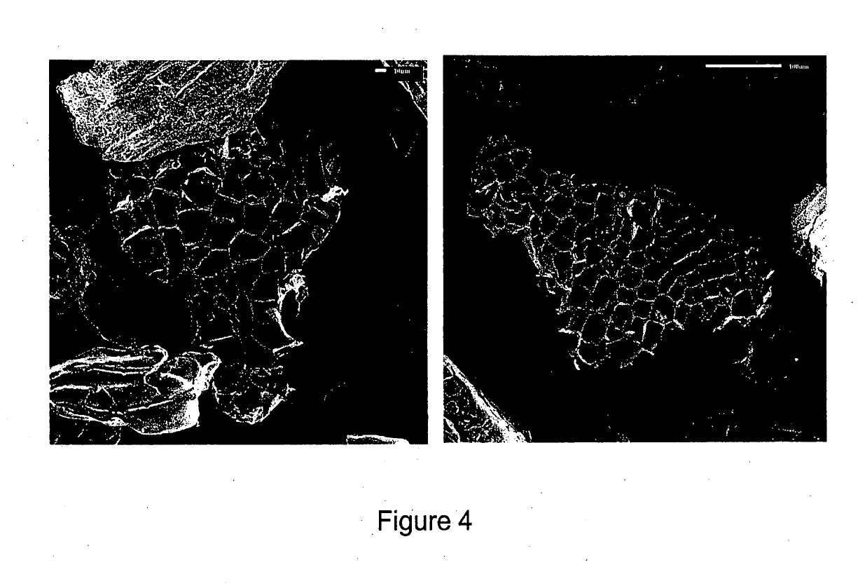

FIGS. 3 and 4 are exemplary scanning electron micrographs of beta-glucan concentrate obtained from barley and oat flour, respectively, processed in accordance with the invention at a pilot plant scale. These micrographs show the effectiveness of the process in creating a honeycomb structure at the pilot plant scale.

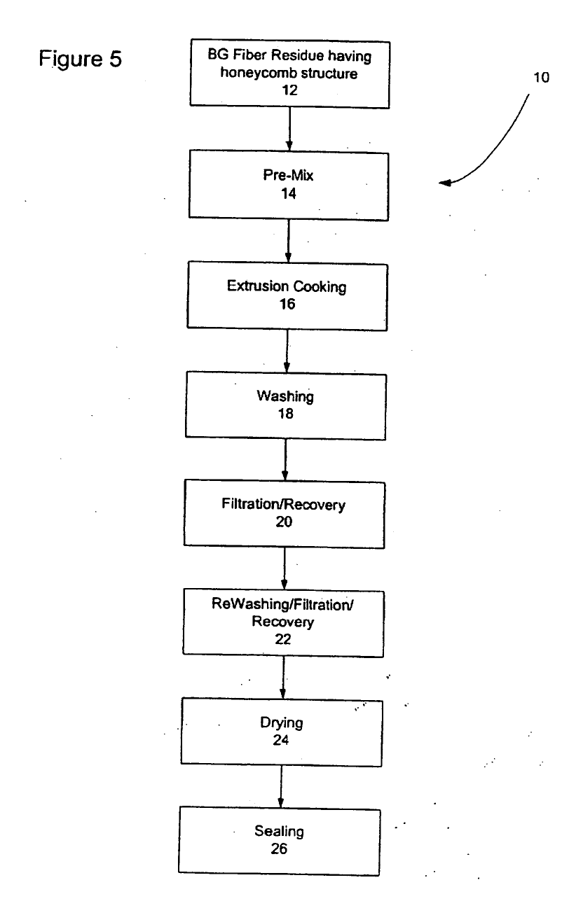

With reference to FIG. 5, a methodology 10 for encapsulating an encapsulant within the beta glucan concentrate or fibre concentrate 12 is described utilizing extrusion technology as one possible approach. Other encapsulating technologies may include spray drying, spray cooling, centrifugal extrusion and inclusion complexation as are known.

With extrusion technology, the fibre concentrate 12 is subjected to a pre-mixing step with water to preferably adjust the moisture content of the fibre concentrate to 15-40% (w/w). Pre-mixing may be performed as is known by mixing the dry fibre concentrate with water with gentle mixing.

The moistened fibre concentrate is then added to an extruder (step 16) and a desired encapsulant is added to the extruder preferably near the inlet so as to ensure maximum mixing and saturation of the fibre concentrate with the encapsulant within the extruder. Alternatively, the encapsulant may be added during the premixing process. The extruder will preferably be operated at conditions wherein partial solubilization of the fibre concentrate will occur in order to enable complete saturation and sealing of encapsulant. Control of variables including moisture content and temperature/heat at the inlet and outlet of the extruder are effective in enhancing encapsulation.

An extrudate from the extruder is collected and will preferably be subjected to various washing 18, filtration and recovery 20 processes (including re-washing, filtration and recovery 22 as appropriate) and a drying 24 process to provide a product having an improved physical stability. A further sealing step 26 may also be incorporated.

The washing step will preferably be a gentle wash and mixing of the extrudate with 50-95% (w/w) aqueous alcohol to dry the extrudate which may be recovered by filtration using a 50 micron screen. The washing step(s) are also effective to remove any encapsulant that is not incorporated within the honeycomb structure.

An alternate encapsulation process includes the steps of preparing a slurry containing a mixture of beta-glucan concentrate, 50% ethanol and encapsulant in a jacketed tank, followed by creating a suitable condition that would lead to partial solubilization of beta-glucan and sealing of the honeycomb structure thus achieving encapsulation. This can be achieved by gradually reducing the ethanol concentration of the slurry by adding water and increasing the temperature of the slurry to an appropriate level in order to trigger partial solubilization of beta-glucan.

Suitable encapsulants would be known to those skilled in the art and may include medical drugs, food ingredients such as flavouring agents, leavening agents, sweeteners, vitamins, minerals, tocols, sterols, omega 3 fatty acids and acidulants or cosmetic ingredients that are prone to oxidative and photo degradation and/or that require delivery at the middle/distal intestinal tract.

Suitable sealants would also be known to those skilled in the art and may include lipid based materials such as mono- and diglycerides which can melt upon heat treatment.

Dispersability Tests

The dispersability of the beta-glucan fiber concentrates having the honeycomb structure was investigated.

Background

One requirement for the use of products as food ingredients is dispersability in water. Fine powders like starch and hygroscopic powders like gums tend to hydrate quickly on the surface and create small lumps. For the majority of industrial applications, premixing with other dry ingredients, or using high shear in-line mixers can eliminate lumping. However, for certain applications, lumping still may pose a problem and, thus it is desirable to utilize products that have high dispersability.

Experimental

A rotator (Roto-Torque, model 7637, Cole-Parmer Instrument Company, Chicago, Ill.) and a water bath was used to measure the dispersability of different beta-glucan powders. Transparent 35 ml tubes are placed on the rotator (tumbler) and filled with a desired amount of water at a set temperature. Beta-glucan samples were placed inside and the tubes were capped and rotated for a desired period of time at predetermined frequency.

In order to conduct experiments at specific temperatures, (for example 37° C.), tubes were set to rotate through the water bath at a slightly higher temperature (typically 0.5-1.0° C.) to compensate for cooling while the tubes rotated through air. To measure the dispersability of powders, a 1% slurry was prepared. A powder sample (200 mg) was weighed into the weighing trays. Tubes (35 ml) were placed on the tumbler and filled with 20 ml of water at 37° C. Beta-glucan powders were placed into tubes and the tubes were immediately capped and rotated for 5 min at 60 Hz. After 5 min, the tumbler was stopped and the contents of the tubes was screened over 2 mm sieve (U.S. mesh 10 equivalent). Tubes were rinsed with 20 ml of warm water at the same temperature and the content of the tubes was gently poured over lumps retained on the screen. Lumps were collected into a pre-weighed tray, and dried overnight at 80° C. After drying, trays with lumps were left to equilibrate for 24 hr and weighed. Dispersability was calculated according to formula:
Dispersability %=[Wtₛₐₘₚₗₑ−Wtₗᵤₘₚₛ]·100/Wtₛₐₘₚₗₑ
where Wtₛₐₘₚₗₑ is the weight of sample (in this case 200 mg) and Wtₗᵤₘₚₛ is the weight of dried and stabilized lumps.

Results

Table 1 compares the dispersability of a commercial 50% oat beta-glucan concentrate prepared in accordance with the prior art by alkali extraction as compared to barley and oat beta-glucan concentrates prepared in accordance with the invention.

The results indicate that a lumping problem exists with the commercial oat product samples whereas the samples prepared in accordance with the invention showed no lumping. Furthermore, whereas the commercial oat beta-glucan samples showed average dispersability of 51%, each of the samples prepared in accordance with the invention exhibited dispersability greater than 99%.

TABLE 1
Dispersability of barley and oat beta-glucan samples prepared in
accordance with the invention compared to a commercial oat
beta-glucan concentrate (200 mg dispersed as 1% slurry).

| | | | Average | |
|---|---|---|---|---|
| | | Lumps | lump | |
| | Lumpiness | weight | weight, | Dispersability |
| Sample | (description) | mg | mg | % ± SD |
| Oat Beta-Glucan | very lumpy | 75 | | |
| concentrate | | | | |
| (Commercial) | | | | |
| Sample 1 | | | | |
| Oat Beta-Glucan | very lumpy | 119 | 97 | 51.5 ± 15.6 |
| concentrate | | | | |
| (Commercial) | | | | |
| Sample 2 | | | | |
| Barley Beta- | no lumps | — | — | >99 |
| Glucan | | | | |
| concentrate | | | | |
| (invention) | | | | |
| Sample 1 | | | | |
| Barley Beta- | no lumps | — | — | >99 |
| Glucan | | | | |
| concentrate | | | | |
| (invention) | | | | |
| Sample 1 | | | | |
| Oat Beta-Glucan | no lumps | — | — | >99 |
| concentrate | | | | |
| (invention) | | | | |
| Sample 1 | | | | |

1. A beta-glucan concentrate having a honeycomb structure.

2. A beta-glucan concentrate as in claim 1 wherein the concentrate is derived from oat or barley flour.

3. A method of preparing a beta-glucan (BG) concentrate product comprising the steps of:
a) mixing a flour and an alcohol to form a flour/alcohol slurry;
b) separating a fiber residue from the alcohol, wherein the fiber residue has a high BG content; and,
c) subjecting the fiber residue from step b) to at least one additional treatment step, the additional treatment step including mixing the fiber residue from step b) with an alcohol to form a fiber residue/alcohol slurry and subjecting the fiber residue/alcohol slurry to a sonication, protease or amylase treatment step or a combination of a sonication, protease or amylase treatment steps and thereafter separating a final fiber concentrate from the fiber residue/alcohol slurry wherein the fiber concentrate has a honeycomb structure.

4. A method as in claim 3 wherein the flour is an oat or a barley flour.

5. The use of a beta-glucan concentrate of claim 1 as wall material for encapsulation.

6. A method of encapsulating a compound within a beta-glucan concentrate having a honeycomb structure comprising the steps of:
a. mixing a beta-glucan concentrate of claim 1 with an encapsulant under conditions to promote occlusion of the encapsulant within the honeycomb structure; and
b. washing the resulting capsules.

7. A method as in claim 6 wherein the conditions of step a) partially solubilizes the honeycomb structure.

8. A method as in claim 6 wherein step a) is an extrusion process.

9. A method as in claim 6 wherein prior to step a), the honeycomb structure is subjected to a pre-mixing step wherein the beta-glucan concentrate is moistened with water.

10. A method as in claim 9 wherein the pre-mixing step adjusts the moisture content of the fibre concentrate to 15-40% (w/w).

11. A method as in claim 5 wherein the capsules are sealed.

12. A method of encapsulating a compound within a beta-glucan concentrate having a honeycomb structure comprising the steps of:
a. mixing a beta-glucan concentrate of claim 1 with an encapsulant under conditions to promote partial solubilization of the beta-glucan concentrate; and,
b. adjusting the conditions of mixing to precipitate beta-glucan concentrate.

13. A method as in claim 12 wherein step a) includes mixing the beta glucan concentrate and encapsulant in ethanol (50% w/w) and step b) includes evaporating the ethanol.

14. A beta-glucan concentrate having a honeycomb structure as shown in FIGS. 1B, 2B, 3 or 4.

15. A beta-glucan concentrate having high dispersability.

16. A beta-glucan concentrate as in claim 15 wherein the dispersability is greater than 52%.

17. A beta-glucan concentrate as in claim 15 wherein the dispersability is greater than 99%.
